(19) United States
(12) Patent Application Publication
(10) Pub. No.: US 2026/0096979 A1
(43) Pub. Date: Apr. 9, 2026

(54) Chemical Compositions for Lip Plumper

(71) Applicant: Mehran Taghdiri, Los Angeles, CA (US)

(72) Inventor: Mehran Taghdiri, Los Angeles, CA (US)

(21) Appl. No.: 18/908,868

(22) Filed: Oct. 8, 2024

(51) Int. Cl. A61K 8/67; A61K 8/19; A61K 8/34; A61K 8/49; A61K 8/891; A61Q 1/00; A61Q 19/00

(52) U.S. Cl. CPC A61K 8/678; A61K 8/19; A61K 8/345; A61K 8/4953; A61K 8/675; A61K 8/891; A61Q 1/00; A61Q 19/00

(57) ABSTRACT

The lip plumper composition described in this patent introduces a novel chemical blend of hydrating, plumping, and nourishing ingredients designed to enhance lip volume while maintaining long-term lip health. The product is formulated using a combination of Botanisil PDH-151, Botanessential Olive S-EHO, Botanol HSO, Vitamin E Nicotinate, and Benzyl Nicotinate, with additional stabilizers such as Aerosil R 974 and a flavor component. The lip and skin treatment composition described in this patent utilizes a two-phase emulsion system to deliver multi-functional cosmetic benefits, including hydration, firmness, and anti-aging effects. The formula combines an oil-based Phase 1, containing Emulsil WO 3155, Euxyl PE 9010, and Vitamin E Acetate, with a water-based Phase 2, containing Water, Propandiol, Sodium Nitrate, Caffeine, Algacktiv Collage, and Botanecuetical.

### BACKGROUND OF THE INVENTION

### Field of Invention

The present invention relates to the field of cosmetic chemistry, particularly focusing on the formulation of lip plumping products designed to enhance lip volume. This invention involves a unique chemical composition of active ingredients, including Botanisil PDH-151, Botanessential Olive S-EHO, Botanol HSO, Aerosil R 974, Vitamin E Nicotinate, and Benzyl Nicotinate, among others, that are combined to create a stable and effective lip plumper. This formula has been specifically designed to provide stability under various environmental conditions while delivering noticeable results in lip volume enhancement.

This invention also focuses on a lip and skin treatment composition containing Phase 1, which is an oil-based premix including Emulsil WO 3155, Euxyl PE 9010, and Vitamin E Acetate, and Phase 2, which is a water-based premix including Water, Propandiol, Sodium Nitrate, Caffeine, Algacktiv Collage, and Botanecuetical. The formulation is designed to provide deep hydration, improve skin firmness, and enhance lip smoothness while delivering antioxidant and anti-aging benefits.

### BRIEF SUMMARY OF THE INVENTION

The lip plumper chemical composition described in this patent is formulated using a combination of ingredients designed to plump the lips by increasing circulation and hydrating the skin for a fuller appearance. The key active ingredient, Botanisil PDH-151, makes up 77.25% of the formula, working synergistically with other components such as Botanessential Olive S-EHO (12%), Botanol HSO (2%), Vitamin E Nicotinate (2%), and Benzyl Nicotinate (4%) to stimulate blood flow and provide long-lasting hydration. The formula is enhanced with 1% flavor to create a pleasant user experience.

The stability of this product under stress conditions, including freeze-thaw cycles and elevated temperatures, sets it apart from existing lip plumping formulations. Testing has shown that the formula remains stable after three cycles of freezing and thawing over three days, as well as being stable for three months at 45 degrees Celsius. This durability is essential for ensuring consistent results and maintaining the efficacy of the product over time, particularly under varying storage and environmental conditions.

Comparatively, many lip plumping products on the market either lack stability under such conditions or do not combine the same percentage of active ingredients, limiting their effectiveness. The inclusion of Botanessential Olive S-EHO and Botanol HSO adds both nourishing properties and contributes to the formula's ability to enhance the lips without causing irritation, which is a common issue with many competing products.

This patent presents a lip and skin treatment composition comprising two distinct phases: an oil-based Phase 1 Premix and a water-based Phase 2 Premix. The combination of these phases results in a stable emulsion that provides a variety of cosmetic benefits, including hydration, firmness, and anti-aging effects

The composition is divided into two phases:

Phase 1 Premix contains 30% Emulsil WO 3155, 1% Euxyl PE 9010, and 1% Vitamin E Acetate. This phase forms the oil portion of the emulsion, offering hydration, skin barrier protection, and preservation.

Phase 2 Premix contains 21% Water, 3% Propandiol, 15% Sodium Nitrate, 4% Caffeine, 20% Algacktiv Collage, and 5% Botanecuetical. This phase delivers moisture, firming benefits, and bioactive ingredients that enhance skin structure and function

This two-phase system is combined to create a homogeneous emulsion that remains stable under different environmental conditions. The result is a cosmetic product that deeply moisturizes the skin and lips, enhances firmness, reduces the appearance of fine lines, and offers antioxidant protection.

This formulation has been tested for stability, remaining effective after exposure to freeze-thaw cycles, and temperatures ranging from 55 degrees Celsius for 3 days to 45 degrees Celsius for 3 months. Its dual-phase composition allows for better absorption of active ingredients and longer-lasting effects on the skin and lips.

### DETAILED DESCRIPTION

### Introduction and Background

Lip plumpers are popular cosmetic products designed to create the appearance of fuller lips without the need for invasive procedures. These products generally work by increasing blood flow to the lips, causing them to swell slightly, or by hydrating the skin to make the lips appear more voluminous. Many lip plumpers contain irritants such as menthol or capsicum to create a tingling sensation, which temporarily enhances the lips'appearance. However, these formulations often lead to irritation, redness, or inconsistent results.

The present invention seeks to address these limitations by offering a stable, irritation-free formula that provides lasting plumping effects while nourishing the lips. The chemical composition in this lip plumper ensures effective results without causing significant discomfort to the user. The addition of hydrating and soothing ingredients, including Botanessential Olive S-EHO and Vitamin E Nicotinate, ensures that the product delivers a smooth, glossy finish while increasing lip volume.

### Key Ingredients and Their Functions

Botanisil PDH-151 (77.25%): A silicone derivative, Botanisil PDH-151 is the primary ingredient responsible for forming a smooth and consistent base in the lip plumper. This compound is known for its hydrating properties and ability to create a protective barrier on the skin, locking in moisture to prevent dryness.

Botanessential Olive S-EHO (12.00%): This ingredient is derived from olives and is rich in squalene, which acts as a powerful emollient. It helps to soften the lips, providing a supple texture and promoting hydration. Olive S-EHO also contributes to the formula's stability by enhancing the spreadability of the product.

Botanol HSO (2.00%): Botanol HSO is a plant-based humectant that attracts moisture to the skin, keeping the lips hydrated throughout the day. In addition to its hydrating effects, this ingredient plays a key role in maintaining the overall viscosity of the formula, ensuring that the product applies evenly.

Vitamin E Nicotinate (2.00%): This form of Vitamin E is known for its anti-inflammatory and antioxidant properties. In this formulation, Vitamin E Nicotinate works to soothe the lips, reduce redness, and improve circulation to provide a plumping effect without irritation.

Benzyl Nicotinate (4.00%): A vasodilator, Benzyl Nicotinate increases blood flow to the lips, resulting in a visible plumping effect. It also promotes the delivery of oxygen and nutrients to the lip tissue, enhancing lip health and appearance.

Aerosil R 974 (1.50%): A form of silica, Aerosil R 974 is used to thicken the formulation and improve its spreadability. It ensures that the product maintains its consistency over time, preventing separation of the ingredients.

Flavor Placeholder (1.00%): The addition of a flavor provides a pleasant sensory experience for the user, masking the chemical smell of the active ingredients and enhancing the overall appeal of the product.

### Mixing and Formulation Process

The preparation of this lip plumper involves a precise mixing process to ensure that all ingredients are evenly distributed and homogeneously blended. The process is as follows:

Combine the Ingredients: All components—Botanisil PDH-151, Botanessential Olive S-EHO, Botanol HSO, Vitamin E Nicotinate, Benzyl Nicotinate, Aerosil R 974, and the flavor—are measured out in their respective proportions.

Mix Until Homogeneous: The ingredients are then mixed in a high-shear mixer for a period of time sufficient to ensure that the mixture is uniform and all particles are fully integrated.

Ensure Stability: The formulation is tested for stability using a series of freeze-thaw cycles and heat exposure tests. The product is frozen and thawed for three cycles over a span of three days, and then it is subjected to 55 degrees Celsius for three days and 45 degrees Celsius for three months. These tests ensure that the formulation remains stable and that there is no phase separation, crystallization, or loss of efficacy

The final product is a smooth, stable lip plumper that can be applied evenly to the lips without separation of ingredients or degradation of its active compounds. Its stability under extreme conditions ensures that it will maintain its effectiveness even after prolonged storage or exposure to varying temperatures.

### Comparative Analysis with Existing Products

### Traditional Lip Plumpers

Most traditional lip plumpers rely on irritating agents such as menthol, capsaicin, or cinnamon to cause a tingling sensation, which leads to temporary swelling of the lips. These formulas often cause discomfort, redness, and dryness, particularly for users with sensitive skin. Moreover, many of these products tend to have short-lived effects, requiring frequent reapplication throughout the day to maintain the desired appearance.

### Comparison to the Current Invention

The lip plumper formula in this patent offers a clear improvement over traditional lip plumpers in several ways:

Stability: One of the primary advantages of this formulation is its stability under various conditions. Most existing products do not undergo rigorous testing for freeze-thaw cycles or exposure to elevated temperatures, which can lead to the breakdown of active ingredients or separation of components. The current invention remains stable for three freeze-thaw cycles and can withstand temperatures as high as 55 degrees Celsius, making it highly durable and reliable.

Irritation-Free Formula: Unlike traditional plumpers that rely on irritants, this formulation uses Vitamin E Nicotinate and Benzyl Nicotinate to stimulate blood flow and provide a plumping effect without causing irritation or discomfort. This makes the product suitable for a broader audience, including those with sensitive skin or a history of allergic reactions to harsher ingredients.

Hydration and Nourishment: The inclusion of Botanessential Olive S-EHO and Botanol HSO provides deep hydration and nourishment to the lips, making them appear fuller and healthier over time. Traditional lip plumpers often focus solely on the plumping effect and neglect the importance of hydration, leading to dryness and peeling after use.

Longer-Lasting Effects: Due to its hydrating properties and stable formulation, this lip plumper provides longer-lasting effects compared to traditional products. Users do not need to reapply the product as frequently, as the plumping and moisturizing benefits are sustained throughout the day.

### Advantages Over Competitor Brands

In comparison to high-end lip plumpers currently available on the market, the chemical composition presented in this patent offers several additional benefits:

Reduced Sensitivity: Many luxury lip plumpers cause significant sensitivity and redness due to the high concentration of irritants used to achieve the plumping effect. The current formulation uses a gentler approach, combining Vitamin E Nicotinate and Benzyl Nicotinate to increase blood flow without causing undue irritation. This allows for daily use without discomfort.

Enhanced Moisturization: Competitor brands often overlook the importance of hydration in their formulas. In contrast, the use of Botanol HSO and Botanessential Olive S-EHO in this formula ensures that the lips are not only plumped but also deeply moisturized. This dual action of plumping and hydration leads to healthier, more supple lips over time, addressing both immediate cosmetic concerns and long-term lip health.

### Extended Product Shelf Life

The stability testing of this lip plumper, which includes rigorous freeze-thaw and high-temperature exposure tests, further demonstrates its resilience compared to many competing products. Many lip plumpers break down or separate after prolonged exposure to heat or freezing conditions, rendering them ineffective over time. In contrast, the current invention maintains its consistency and efficacy, even under extreme storage conditions. This extended shelf life is a crucial selling point, particularly for retail environments where products may face inconsistent temperatures during shipping and storage.

### Key Features of the Lip Plumper Composition

The innovative aspects of this formulation lie in both its carefully chosen ingredients and its ability to remain stable and effective under adverse conditions. Each ingredient plays a vital role in delivering the desired effects while ensuring that the product remains usable and pleasant for consumers over extended periods.

### Botanisil PDH-151 (77.25%)

The base of this formulation, Botanisil PDH-151, is a silicone-based compound that ensures the smooth texture and spreadability of the lip plumper. The primary function of this ingredient is to provide a barrier on the lips, locking in moisture while preventing external irritants from causing dryness or cracking. Its silicone base also imparts a glossy finish, adding to the aesthetic appeal of the product.

### Botanessential Olive S-EHO (12%)

Derived from olives, Botanessential Olive S-EHO is rich in squalene, which is known for its ability to mimic the skin's natural oils, providing long-lasting hydration without leaving a greasy residue. Squalene has been extensively studied for its ability to reduce moisture loss and improve skin elasticity, making it a perfect addition to a lip care product. This ingredient not only helps keep the lips moisturized but also contributes to the longevity of the plumping effect by maintaining skin suppleness.

### Botanol HSO (2%)

As a potent humectant, Botanol HSO helps attract moisture to the lips, keeping them hydrated for extended periods. This ingredient is particularly effective in dry or harsh climates where the lips are prone to chapping and cracking. By maintaining moisture levels, Botanol HSO helps reduce the frequency of reapplication and ensures that the lips remain smooth and comfortable throughout the day.

### Vitamin E Nicotinate (2%)

Vitamin E Nicotinate is an innovative addition to this formula. While Vitamin E is well known for its antioxidant properties, the nicotinate form enhances blood flow, creating a natural plumping effect without the irritation caused by more aggressive ingredients such as menthol or capsicum. This ingredient not only contributes to the appearance of fuller lips but also provides protection against oxidative stress, ensuring that the lips remain healthy and vibrant even with regular use.

### Benzyl Nicotinate (4%)

As a vasodilator, Benzyl Nicotinate plays a crucial role in increasing circulation to the lips, resulting in a visible plumping effect. Unlike traditional irritants, Benzyl Nicotinate works by gently dilating blood vessels, promoting natural lip enhancement without causing significant redness or discomfort. This ingredient also aids in delivering essential nutrients and oxygen to the lip tissue, promoting long-term lip health.

### Aerosil R 974 (1.5%)

Aerosil R 974 is a form of silica that is used to improve the consistency and spreadability of the product. This ingredient ensures that the lip plumper remains smooth and easy to apply, while also preventing the separation of ingredients over time. The silica helps to maintain the product's structure, even after extended periods of storage or use.

### Flavor Placeholder (1%)

To enhance the user experience, the formula includes 1% flavor, which can be customized based on consumer preferences. The flavor serves to mask any unpleasant odors from the active ingredients while adding to the overall appeal of the product. Common flavors might include vanilla, mint, or fruit-based options, though the formulation can be adapted to accommodate different markets or consumer demands.

### Stability Testing and Results

Stability is a critical factor in cosmetic formulations, especially in products intended for retail sale and consumer use over extended periods. The lip plumper composition in this patent has undergone a series of rigorous tests to ensure that it remains stable and effective under various conditions.

### Freeze-Thaw Testing

The product was subjected to three cycles of freezing and thawing over three days. In each cycle, the product was frozen at −18 degrees Celsius for 24 hours, then thawed at room temperature for 24 hours. The purpose of this test was to simulate the conditions the product might encounter during transportation or storage in fluctuating climates.

Results: After three cycles of freezing and thawing, the product showed no signs of separation, crystallization, or loss of efficacy. The texture and spreadability remained consistent, and the plumping effect was unchanged.

### Heat Stability Testing

To test the product's stability under high temperatures, it was exposed to 55 degrees Celsius for three days and 45 degrees Celsius for three months. These tests simulate the conditions the product might face if stored in a hot environment, such as a warehouse or a delivery vehicle during summer months.

Results: After both heat exposure tests, the product remained stable, with no visible changes in texture or appearance. The active ingredients maintained their efficacy, and the product continued to deliver the desired plumping and moisturizing effects.

These stability results are a testament to the durability of the formula, making it suitable for both retail environments and everyday consumer use. Many competing products fail to meet such stringent stability requirements, which can lead to a breakdown in efficacy or an unpleasant texture after prolonged exposure to extreme conditions.

### 6. Comparative Case Studies with Competitor Products

To further highlight the superiority of this invention, a series of comparative case studies were conducted, testing the current formula against two popular lip plumping products currently available on the market. These products were chosen based on their widespread use and reputation for delivering plumping effects.

### Competitor A

Competitor A is a well-known lip plumper that uses a combination of menthol and cinnamon oil to achieve its plumping effects. The product causes a strong tingling sensation upon application, which results in a temporary increase in lip volume.

Key Findings: While Competitor A produced visible results within minutes of application, the plumping effect lasted only about 30 minutes before fading. Additionally, users reported significant discomfort due to the tingling sensation, with many experiencing redness and irritation.

Comparison: In contrast, the lip plumper described in this patent provides a longer-lasting plumping effect without causing irritation. The combination of Vitamin E Nicotinate and Benzyl Nicotinate ensures a gentle, natural plumping effect that lasts for several hours, without the need for frequent reapplication. Moreover, the hydrating properties of Botanol HSO and Botanessential Olive S-EHO provide additional benefits that Competitor A lacks, such as improved lip health and moisture retention.

### Competitor B

Competitor B is a luxury lip plumper that relies on a high concentration of hyaluronic acid to temporarily increase lip volume. While the product does not cause the same level of irritation as Competitor A, it requires frequent reapplication throughout the day to maintain the desired effect.

Key Findings: Competitor B was able to produce a noticeable plumping effect, but users found that they needed to reapply the product every 1-2 hours to maintain the effect. Additionally, the product tended to leave a sticky residue on the lips, which was uncomfortable for many users.

Comparison: The lip plumper in this patent provides more sustained results, reducing the need for frequent reapplication. Thanks to the inclusion of Botanol HSO and Aerosil R 974, the formula spreads smoothly without leaving a sticky residue. The product also offers a more comprehensive solution, combining plumping, moisturizing, and nourishing properties in one formula, which Competitor B does not achieve as effectively.

### Applications in the Cosmetic Industry

The lip plumper composition described in this patent is versatile and can be applied to a wide range of cosmetic products, including:

Standalone Lip Plumper: The formula can be marketed as a standalone lip plumper in both standard and travel sizes. Its stability and efficacy make it suitable for use in various climates and environments.

Tinted Lip Plumpers: The formula can be adapted to include tints or pigments, creating a combination product that both plumps and adds color to the lips. This would appeal to consumers looking for an all-in-one solution for lip care and makeup.

Lip Care Kits: The product can be included in lip care kits, along with complementary items such as lip scrubs or balms. The inclusion of nourishing ingredients like Botanessential Olive S-EHO makes this formula ideal for pairing with other lip care products.

In the cosmetic industry, the demand for multi-functional products that deliver hydration, anti-aging effects, and overall skin improvement continues to grow. Lip and skin treatment products that provide lasting hydration and enhance skin elasticity are particularly sought after, as consumers look for products that not only improve their appearance but also promote skin health.

Traditional lip and skin care products often rely on single-phase formulations that deliver limited benefits. These products may provide temporary hydration or surface-level improvements but lack the ability to deliver active ingredients deep into the skin, where they are most needed.

The present invention addresses these shortcomings by utilizing a two-phase emulsion system, which combines the benefits of an oil-based phase and a water-based phase. This system allows for better penetration of active ingredients and provides a more stable formulation, delivering longer-lasting effects on both the lips and skin.

### Phase 1 Premix (Oil-Based)

The Phase 1 Premix contains three key ingredients:

Emulsil WO 3155 (30%): A water-in-oil emulsifier that forms the backbone of the oil-based phase. Emulsil WO 3155 helps to stabilize the overall emulsion and ensures that the oil and water phases mix effectively.

Euxyl PE 9010 (1%): A preservative that protects the formula from microbial contamination, ensuring the product's safety and longevity.

Vitamin E Acetate (1%): A potent antioxidant that protects the skin and lips from free radicals, reducing the visible signs of aging and promoting overall skin health.

### Phase 2 Premix (Water-Based)

The Phase 2 Premix contains water-soluble ingredients that provide hydration, firming benefits, and anti-aging effects:

Water (21%): The primary solvent in the water phase, providing essential hydration to the skin and lips.

Propandiol (3%): A humectant that attracts moisture to the skin, keeping it hydrated and preventing dryness.

Sodium Nitrate (15%): A skin-conditioning agent that enhances skin texture and improves elasticity.

Caffeine (4%): Known for its ability to reduce puffiness and firm the skin, caffeine helps to improve the appearance of both lips and skin.

Algacktiv Collage (20%): A marine-derived ingredient that promotes collagen production, enhancing skin firmness and reducing the appearance of fine lines.

Botanecuetical (5%): A plant-based extract with bioactive properties, Botanecuetical helps to soothe the skin, reduce inflammation, and promote overall skin health.

This two-phase system is designed to be mixed until homogeneous, forming a stable emulsion that can be applied to the lips and skin to deliver multiple cosmetic benefits.

### Mixing and Formulation Process

The formulation of the lip and skin treatment composition follows a step-by-step mixing process that ensures the ingredients from both phases are evenly distributed and form a stable emulsion. The detailed process is as follows:

### Phase 1 Preparation

Emulsil WO 3155, Euxyl PE 9010, and Vitamin E Acetate are combined in the oil phase and mixed using moderate heat to fully integrate the ingredients. The temperature during this phase is maintained at approximately 45-50 degrees Celsius to ensure that the ingredients are fully dissolved.

### Phase 2 Preparation

Water, Propandiol, Sodium Nitrate, Caffeine, Algacktiv Collage, and Botanecuetical are mixed together in the water phase. This phase is also heated to ensure the solubility of the active ingredients, particularly the sodium nitrate and caffeine.

### Emulsion Formation

Once both phases are prepared, the oil phase (Phase 1) is slowly added to the water phase (Phase 2) with constant stirring. A high-shear mixer is used to ensure the formation of a stable water-in-oil emulsion. The mixing process continues until the emulsion is homogeneous and all ingredients are fully integrated.

### Stability Testing

The final emulsion is tested for stability by subjecting it to freeze-thaw cycles and heat exposure tests. The product is frozen and thawed for three cycles over three days and is then tested at 55 degrees Celsius for three days and 45 degrees Celsius for three months. These tests ensure that the emulsion remains stable and effective under varying environmental conditions.

### Comparative Analysis with Existing Products: Traditional Single-Phase Formulations

Many traditional lip and skin care products rely on single-phase formulations, which combine oil and water-based ingredients into a single emulsion. While these formulations can provide surface-level hydration or temporary firming effects, they often lack the ability to deliver active ingredients deep into the skin.

Additionally, single-phase formulations are often less stable than two-phase emulsions, leading to separation of ingredients over time or degradation of active compounds. This instability can result in reduced efficacy and shorter shelf life, limiting the long-term benefits of the product.

### Comparison to the Current Invention

The two-phase emulsion system presented in this patent offers several advantages over traditional single-phase formulations:

Stability: The use of Emulsil WO 3155 as the emulsifier in Phase 1 ensures that the oil and water phases mix effectively, forming a stable emulsion. Stability testing has shown that the formulation remains effective even after exposure to freeze-thaw cycles and high temperatures, whereas many single-phase formulations may break down or separate under similar conditions.

Enhanced Delivery of Active Ingredients: The two-phase system allows for better penetration of active ingredients into the skin and lips. The water-based phase (Phase 2) delivers hydrating and firming agents, while the oil-based phase (Phase 1) locks in moisture and provides antioxidant protection. This dual-action system ensures that the product provides both immediate and long-lasting benefits.

Hydration and Firmness: The combination of Algacktiv Collage and Sodium Nitrate in Phase 2 promotes collagen production and improves skin elasticity, making the lips and skin appear firmer and more youthful. These benefits are enhanced by the presence of Caffeine, which helps reduce puffiness and further firms the skin. Traditional formulations often lack this level of synergy between ingredients, resulting in less noticeable improvements in skin texture and firmness.

Gentle on Sensitive Skin: The inclusion of Botanecuetical, a plant-based bioactive ingredient, helps to soothe the skin and reduce inflammation, making this formulation suitable for sensitive skin types. Many traditional formulations rely on harsher ingredients that can cause irritation or redness, particularly in individuals with delicate skin.

### Advantages Over Competitor Products

In comparison to high-end lip and skin treatments currently available on the market, the chemical composition in this patent offers several additional advantages:

Better Absorption: Many competitor products do not achieve the same level of absorption as the two-phase emulsion system presented in this invention. The use of both an oil-based and water-based phase allows for the deeper penetration of active ingredients into the skin and lips. The water phase delivers hydrating ingredients like Propandiol and Algacktiv Collage, which are absorbed quickly to provide immediate moisture, while the oil phase forms a protective barrier that locks in this moisture, preventing dehydration throughout the day. Competitor products, especially single-phase formulations, often provide only superficial hydration that fades within a few hours, requiring frequent reapplication.

Longer-Lasting Effects: The dual-phase system offers longer-lasting results than traditional single-phase lip and skin treatments. By combining the firming benefits of Sodium Nitrate and Caffeine with the protective qualities of Vitamin E Acetate and Emulsil WO 3155, this formula ensures that the lips and skin remain hydrated, firm, and smooth for extended periods. This reduces the need for frequent application and makes the product more convenient for consumers.

Improved Stability and Shelf Life: One of the most significant advantages of this formulation over competing products is its stability. Through rigorous stability testing, including freeze-thaw cycles and high-temperature exposure, this product has demonstrated that it can withstand extreme conditions without degrading. Many other lip and skin care products may experience phase separation or ingredient breakdown when exposed to similar conditions, reducing their efficacy and shortening their shelf life.

Synergistic Ingredient Combination: While competitor products may include a mix of hydrating or anti-aging ingredients, they often do not leverage the synergy between water-soluble and oil-soluble ingredients as effectively as this invention. The use of Algacktiv Collage, Caffeine, and Sodium Nitrate in the water phase works in concert with Vitamin E Acetate and Emulsil WO 3155 in the oil phase to provide comprehensive skin benefits that include hydration, firmness, antioxidant protection, and long-term improvement in skin texture.

### Key Features of the Lip and Skin Treatment Composition

The chemical composition of the lip and skin treatment detailed in this patent offers several unique and innovative features that set it apart from existing products on the market. These features are designed to ensure maximum efficacy, long-lasting results, and ease of use for consumers seeking premium lip and skin car

### Phase 1 Premix (Oil-Based)

Emulsil WO 3155 (30%): This emulsifier is critical to forming a stable water-in-oil emulsion, which enhances the texture and application of the product. Emulsil WO 3155 allows for the smooth incorporation of oil-soluble and water-soluble ingredients, ensuring that the final product is both stable and effective. Its ability to bind water and oil phases ensures that the active ingredients in both phases are delivered to the skin evenly and efficiently.

Euxyl PE 9010 (1%): This broad-spectrum preservative is added to protect the formulation from microbial contamination, ensuring the product's safety and longevity. The presence of Euxyl PE 9010 in the oil phase helps to preserve the product during both storage and usage, preventing the growth of bacteria and fungi that could otherwise compromise the formula

Vitamin E Acetate (1%): As a well-known antioxidant, Vitamin E Acetate provides essential protection against environmental stressors, such as UV rays and pollution. Its presence in the oil phase helps to neutralize free radicals, preventing oxidative damage to the lips and skin. Additionally, Vitamin E improves the skin's barrier function, ensuring that moisture is locked in and that the skin remains hydrated throughout the day

### Phase 2 Premix (Water-Based)

Water (21%): This phase serves as the main hydrating component of the formula, delivering essential moisture to the skin. The water phase allows for the delivery of other water-soluble ingredients, such as Caffeine and Sodium Nitrate, ensuring that they penetrate the skin effectively.

Propandiol (3%): A powerful humectant, Propandiol attracts and retains moisture in the skin. Its ability to draw moisture from the surrounding environment ensures that the skin remains hydrated even in dry conditions. Unlike some humectants that can leave the skin feeling sticky or greasy, Propandiol absorbs quickly, leaving the skin feeling soft and smooth.

Sodium Nitrate (15%): This ingredient plays a vital role in improving skin texture and elasticity. Sodium Nitrate works by promoting collagen synthesis, which helps to firm the skin and reduce the appearance of fine lines and wrinkles. Its presence in the water phase ensures that the skin remains firm and youthful, providing both immediate and long-term anti-aging benefits.

Caffeine (4%): Known for its ability to reduce puffiness and improve circulation, Caffeine is a key active ingredient in this formula. By stimulating blood flow to the skin, Caffeine helps to reduce the appearance of dark circles, fine lines, and other signs of aging. Its presence in the water phase allows for easy absorption into the skin, providing both firming and brightening effects.

Algacktiv Collage (20%): This marine-derived ingredient is rich in collagen-boosting compounds, which help to improve skin firmness and elasticity. Algacktiv Collage works by stimulating the skin's natural collagen production, resulting in smoother, firmer, and more youthful-looking skin. Its inclusion in the water phase ensures that it is easily absorbed and delivered deep into the skin, where it can provide maximum benefits.

Botanecuetical (5%): A plant-based bioactive ingredient, Botanecuetical offers anti-inflammatory and soothing properties, making this formula suitable for sensitive skin. Its ability to reduce redness and irritation ensures that the product can be used by individuals with all skin types, including those prone to sensitivity or allergic reactions. Additionally, Botanecuetical enhances the overall health of the skin by promoting the regeneration of healthy skin cells.

### Stability Testing and Results

The lip and skin treatment composition in this patent has been subjected to a series of rigorous stability tests to ensure that it remains effective and usable over time. Stability is an essential factor in cosmetic formulations, particularly for products that are sold in retail environments and used by consumers over extended periods.

### Freeze-Thaw Testing

The product was exposed to three cycles of freezing and thawing over a period of three days. In each cycle, the product was frozen at −18 degrees Celsius for 24 hours and then thawed at room temperature for 24 hours. This testing simulates the conditions the product might face during shipping or storage in climates where temperatures fluctuate.

Results: After three freeze-thaw cycles, the product remained stable, with no phase separation, crystallization, or loss of efficacy. The emulsion maintained its smooth texture and consistent application, and the active ingredients were unaffected by the freeze-thaw process.

### Heat Stability Testing

The product was also tested at 55 degrees Celsius for three days and 45 degrees Celsius for three months. These tests simulate the conditions the product may encounter during storage in hot environments, such as warehouses or shipping vehicles during summer months.

Results: After both heat exposure tests, the product showed no signs of degradation or separation. The emulsion remained stable, and the active ingredients continued to deliver their intended benefits, including hydration, firming, and antioxidant protection.

The results of these stability tests demonstrate that the lip and skin treatment composition is highly durable and can withstand a range of environmental conditions. This ensures that the product will remain effective even after prolonged storage or exposure to extreme temperatures, making it ideal for both retail and consumer use.

### 6. Comparative Case Studies with Competitor Products

### Competitor A

Competitor A is a popular single-phase lip and skin treatment that uses hyaluronic acid as its primary hydrating ingredient. While the product delivers noticeable hydration upon application, it lacks the firming and antioxidant benefits offered by the formulation described in this patent.

Key Findings: Competitor A provides temporary hydration, but users reported that the effects faded within a few hours. Additionally, the product did not offer any firming or anti-aging benefits, limiting its overall efficacy.

Comparison: The lip and skin treatment in this patent offers longer-lasting hydration due to the combination of Propandiol and Algacktiv Collage. The firming effects of Caffeine and Sodium Nitrate further enhance the product's benefits, providing a more comprehensive solution for individuals seeking both hydration and anti-aging effects.

### Competitor B

Competitor B is a luxury skin treatment that focuses on reducing fine lines and wrinkles. The product contains collagen-boosting ingredients, but its single-phase formulation limits its ability to deliver deep hydration.

Key Findings: Competitor B was effective in reducing the appearance of fine lines, but users found that the product was not hydrating enough, especially for individuals with dry or sensitive skin. The product also required frequent reapplication to maintain its effects.

Comparison: The two-phase emulsion system in this patent ensures deep hydration while promoting collagen production and firming the skin.

Unlike Competitor B, which focuses solely on anti-aging benefits, this product offers a holistic approach that addresses hydration, firmness, and overall skin health. The dual-phase formulation also allows for longer-lasting effects, longer-lasting effects, ensuring that users do not need to reapply the product as frequently. Additionally, the inclusion of Botanecuetical provides soothing and anti-inflammatory benefits that make the product suitable for sensitive skin, which many luxury anti-aging treatments may lack.

### Advantages Over Competitor Products

The lip and skin treatment composition described in this patent provides several distinct advantages over other competing products:

Enhanced Hydration: While some competitor products focus primarily on hydration or anti-aging, this formulation delivers both. The combination of Propandiol, Algacktiv Collage, and Botanol HSO ensures deep, long-lasting moisture. These ingredients, combined with the dual-phase system, prevent dehydration and keep lips and skin hydrated throughout the day.

Firming and Collagen Production: Few products on the market offer the potent combination of Sodium Nitrate and Algacktiv Collage, which work synergistically to improve skin elasticity and promote natural collagen synthesis. These ingredients target the skin's structural integrity, providing immediate firmness and long-term anti-aging benefits. The results are more noticeable and longer-lasting compared to products that rely solely on hyaluronic acid or other single-function ingredients.

Reduced Sensitivity: Many high-end lip and skin treatments are not formulated for individuals with sensitive skin. In contrast, the presence of Botanecuetical ensures that this product is safe for all skin types, including those prone to irritation or inflammation. The gentle, plant-based bioactive compounds in Botanecuetical calm the skin and reduce redness, making the product a versatile solution for a wide range of users.

Multi-Functional Benefits: Unlike many competitor products that focus on a single benefit, such as hydration or anti-aging, this lip and skin treatment offers multi-functional benefits. It hydrates, firms, soothes, and protects the skin and lips, making it a comprehensive solution for overall skin health. Users can experience improvements in both texture and appearance, with results that are sustained over time.

### Applications in the Cosmetic Industry

The lip and skin treatment composition described in this patent is a versatile product that can be used in a variety of cosmetic applications, including:

Stand-Alone Lip Treatment: This formula can be marketed as a stand-alone lip treatment designed to plump, hydrate, and protect the lips. The dual-phase system ensures that the lips receive both hydration and nourishment while also providing a visible plumping effect due to the firming action of Caffeine and Algacktiv Collage.

Anti-Aging Skin Treatment: The product can be marketed as an anti-aging skin treatment for both the lips and surrounding skin. The presence of Sodium Nitrate, Vitamin E Acetate, and Algacktiv Collage ensures that the formula reduces the appearance of fine lines, firms the skin, and promotes long-term collagen production.

Multipurpose Skin Care Kit: This product can be included in multi-step skin care kits designed to provide comprehensive care for the lips and skin. The formula can be paired with complementary products, such as exfoliants or serums, to create a complete skin care regimen that addresses a variety of concerns, including hydration, firmness, and anti-aging.

Tinted Lip Balms and Treatments: The formula can also be adapted to include tints or pigments, creating a tinted lip balm that provides both cosmetic and treatment benefits. The inclusion of pigments allows the product to function as both a lip treatment and a beauty product, offering color alongside the moisturizing and firming benefits.

What is claimed is:

1. A lip plumping composition, comprising:
a. 77.25% Botanisil PDH-151,
b. 12.00% Botanessential Olive S-EHO,
c. 2.00% Botanol HSO,
d. 0.25% Botanessential RRST,
e. 1.50% Aerosil R 974,
f. 2.00% Vitamin E Nicotinate,
g. 4.00% Benzyl Nicotinate,
h. 1.00% Flavor Placeholder,
i. wherein the ingredients are combined and mixed until homogeneous, forming a stable lip plumping composition.

2. The lip plumping composition as claimed in claim 1, wherein the composition is stable under the following conditions:
a. Freeze-thaw cycles for 3 cycles over 3 days,
b. Exposure to 55 degrees Celsius for 3 days,
c. Exposure to 45 degrees Celsius for 3 months.

3. AThe lip plumping composition as claimed in claim 1, wherein Botanisil PDH-serves as a silicone-based compound that provides a smooth, moisturizing layer on the lips, locking in hydration and forming a protective barrier.

4. The lip plumping composition as claimed in claim 1, wherein Botanessential Olive S-EHO functions as an emollient, rich in squalene, which enhances lip hydration, providing a soft texture to the lips without a greasy residue.

5. The lip plumping composition as claimed in claim 1, wherein Botanol HSO acts as a humectant, attracting moisture to the lips and ensuring sustained hydration throughout the day.

6. The lip plumping composition as claimed in claim 1, wherein Vitamin E Nicotinate and Benzyl Nicotinate work synergistically to promote blood flow, thereby increasing lip volume without causing irritation.

7. The lip plumping composition as claimed in claim 1, wherein Aerosil R 974 thickens the formula and ensures the ingredients remain evenly distributed, preventing separation over time.

8. The lip plumping composition as claimed in claim 1, wherein the composition is designed to avoid the irritation commonly caused by traditional lip plumpers containing menthol or capsicum, providing a gentle but effective plumping action.

9. The lip plumping composition as claimed in claim 1, wherein the composition is formulated to be applied as a standalone lip plumper, with optional variations including tinted formulations or inclusion in lip care kits.

10. The method of making the lip plumping composition as claimed in claim 1, comprising:
a. Measuring each ingredient in its specified percentage,
b. Combining the ingredients,
c. Mixing the ingredients using a high-shear mixer until a homogeneous mixture is formed,
d. Ensuring that the composition passes stability testing under freeze-thaw and heat conditions, confirming no phase separation or loss of efficacy.

11. A lip and skin treatment composition, comprising:
a. Phase 1 Premix, consisting of:
i. 30% Emulsil WO 3155,
ii. 1% Euxyl PE 9010,
iii. 1% Vitamin E Acetate,
b. Phase 2 Premix, consisting of:
i. 21% Water,
ii. 3% Propandiol,
iii. 15% Sodium Nitrate,
iv. 4% Caffeine,
v. 20% Algacktiv Collage,
vi. 5% Botanecuetical,
vii. wherein the ingredients are combined and mixed until homogeneous, forming a stable water-in-oil emulsion suitable for lip and skin treatment.

12. The lip and skin treatment composition as claimed in claim 11, wherein Emulsil WO 3155 serves as the primary emulsifier, ensuring the stability and homogeneity of the water-in-oil emulsion.

13. The lip and skin treatment composition as claimed in claim 11, wherein Euxyl PE 9010 acts as a preservative, preventing microbial contamination and ensuring product safety during storage and use.

14. The lip and skin treatment composition as claimed in claim 11, wherein Vitamin E Acetate provides antioxidant protection, reducing oxidative damage to the lips and skin while promoting overall skin health.

15. The lip and skin treatment composition as claimed in claim 11, wherein Propandiol acts as a humectant, attracting moisture to the skin and keeping it hydrated for extended periods.

16. The lip and skin treatment composition as claimed in claim 11, wherein Sodium Nitrate promotes collagen synthesis and improves skin elasticity, resulting in firmer, more youthful-looking skin.

17. The lip and skin treatment composition as claimed in claim 11, wherein Caffeine reduces puffiness and improves circulation, enhancing skin firmness and reducing the appearance of fine lines.

18. The lip and skin treatment composition as claimed in claim 11, wherein Algacktiv Collage stimulates collagen production, further improving skin elasticity and reducing the visible signs of aging.

19. The lip and skin treatment composition as claimed in claim 11, wherein Botanecuetical provides anti-inflammatory and soothing properties, making the product suitable for sensitive skin.

20. A method of making the lip and skin treatment composition, comprising:
a. Preparing Phase 1 by mixing Emulsil WO 3155, Euxyl PE 9010, and Vitamin E Acetate under moderate heat until homogeneous,
b. Preparing Phase 2 by mixing Water, Propandiol, Sodium Nitrate, Caffeine, Algacktiv Collage, and Botanecuetical under moderate heat until homogeneous,
c. Slowly adding Phase 1 to Phase 2 while stirring to form a water-in-oil emulsion, using a high-shear mixer to ensure the phases are fully integrated and the emulsion is stable.
